(19) United States
(12) Patent Application Publication
(10) Pub. No.: US 2014/0377196 A1
(43) Pub. Date: Dec. 25, 2014

(54) Customizable topical skin care product with variable product to ingredient ratio

(71) Applicant: Rizwan Watto, Chicago, IL (US)

(72) Inventor: Rizwan Watto, Chicago, IL (US)

(21) Appl. No.: 13/923,323

(22) Filed: Jun. 20, 2013

(51) Int. Cl. A61K 8/02; A61Q 5/00; A61Q 19/10; A61Q 19/08; A61Q 9/04; A61Q 17/00; A61Q 9/02; A61Q 7/00; A61Q 5/12; A61Q 5/02; A61K 8/96; A61Q 17/04

(52) U.S. Cl. CPC A61K 8/0204; A61K 8/965; A61Q 5/002; A61Q 19/10; A61Q 19/08; A61Q 17/04; A61Q 17/00; A61Q 9/02; A61Q 7/00; A61Q 5/12; A61Q 5/02; A61Q 9/04
USPC 424/59; 424/70.1; 424/73; 514/769; 510/119; 510/137; 510/159; 8/161; 206/219

(57) ABSTRACT

A user controlled, customizable topical skin care product comprising a topical skin care product, at least one separate ingredient that may be combined with said product, and a means for combining said product and said ingredient. This device allows users to customize the skin care product's product-to-ingredient ratio.

### BACKGROUND OF INVENTION

User customizable topical skin care product consisting of a topical skin care product along with a separate ingredient that may be combined with said skin care product so the user can combine the ingredient with the skin care product and create a desired product-to-ingredient ratio.

### FIELD OF INVENTION

This application relates to topical skin care products, specifically to an improved skin care product.

### BACKGROUND OF INVENTION

### Discussion of Prior Art

Topical skin care products have been available as uniform, already-mixed, finished products with fixed ingredient ratios. This means that users have not been able to safely or easily modify properties of the skin care product they are using. This presents a problem for users who want their skin care product to have an additional amount of a certain ingredient in order to obtain the benefits of that ingredient. In the past if a user wanted a skin care product to provide extra lubrication, exfoliation, or some other property the user would have had to purchase a different skin care product or use more of an existing product. Having to purchase a different skin care product can be problematic, because finding and obtaining said product could be time consuming and frustrating. Moreover, the additional cost can be a burden as well. Using a greater amount of a skin care product just to gain the benefits of one of its qualities can be burdensome as well, because it is wasteful and it means having to expose the skin to all the other ingredients of the product in order to gain the benefits of just one or few ingredients. Another problem with the prior art is a person's skin may require one ratio of a topical skin care product to ingredient one day and a different one the next. Different parts of the body may require different ratios as well. Sadly the prior art does not give users the benefit of being able to easily alter the properties of their skin care products. This presents a problem for those who do not wish to purchase a variety of products for their various skin care needs to use on different occasions or different parts of the body. To be able to give the user the ability to easily customize a skin care product would free the user from the inadequacies of formerly available skin care products. A solution to this problem is necessary to allow people the freedom from having to purchase several different topical skin care products.

### SUMMARY

In accordance with one embodiment, a customizable topical skin care product comprising a topical skin care product along with at least one separate ingredient that may be mixed with the skin care product in order to allow the user to alter the product-to-ingredient ratio. The skin care product may consist of a cleanser, moisturizer, toner, soap, or some other topical product that may be used to care for the skin. The separate ingredient may consist of some oil, sea salt, or some other element or mixture. The user is given instructions that encourage him to mix a desired amount of product with a desired amount of ingredient before use. Providing the user with instructions, a skin care product, and a separate ingredient enables the user to create a customized solution. The user may add a desired amount of the separate ingredient to the skin care product before every use, thus allowing for a variety of different product-ingredient ratios.

### DRAWINGS

### List of Reference Numerals

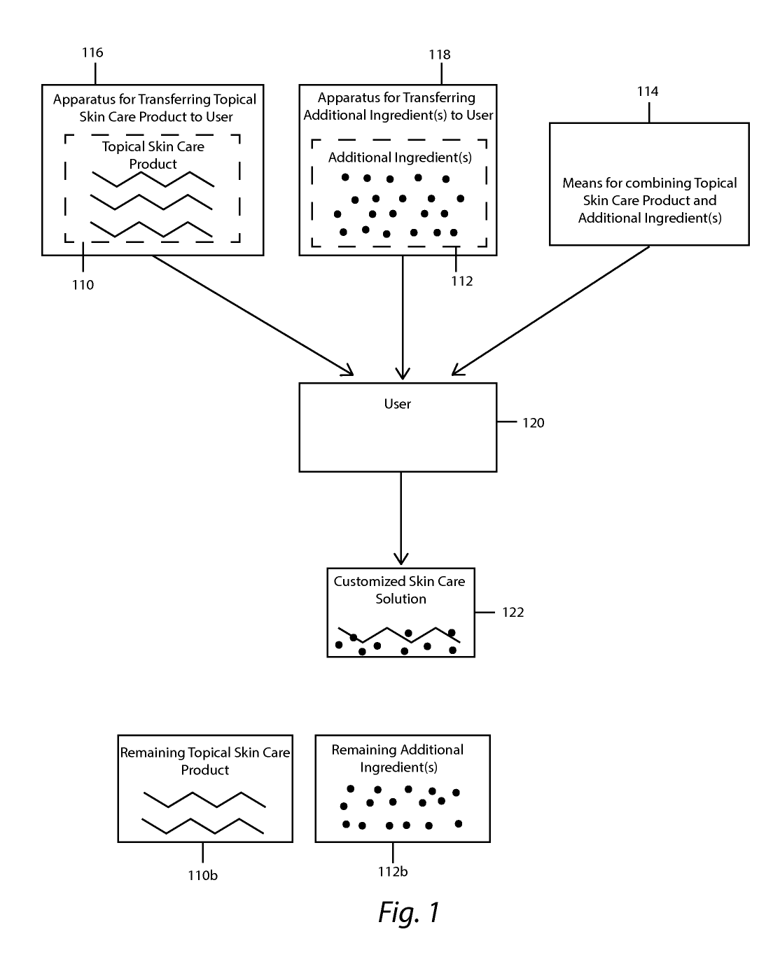

FIG. 1 is a diagram illustrating one embodiment of the present device.

110a—Topical skin care product
112a—Separate ingredient
114—Means for combining said topical skin care product (110) and said separate ingredient (112)
116—Apparatus for transferring said topical skin care product to user (110)
118—Apparatus for transferring said separate ingredient to user (112)
120—User
122—Customized skin care solution
110b—Remaining separate ingredient, left unaltered for future use
112b—Remaining topical skin care product, left unaltered for future use

### DETAILED DESCRIPTION

### First Embodiment—FIGS. 110-122

FIG. 1 shows a representation of one embodiment of the present device. Element 110a represents a topical skin care product, which in this embodiment refers specifically to a liquid facial cleanser or liquid soap. Element 110b represents any leftover liquid soap after a desired amount of said soap 110a is used to create a custom solution 122. Element 112a represents separate ingredient, which in this embodiment refers specifically to sea salt. Element 112b represents any left over sea salt after a desired amount of said sea salt 112a is used to create a custom solution 122. Element 114 represents a means for a user to combine said Skin Care Product 110a and said Separate Ingredient 112a, which in this embodiment refers specifically to a set of instructions that show the user how to combine the facial cleanser 110a and the sea salt 112a. Element 120 represents a user, defined as an individual who wishes to administer said product 110a and said ingredient 112a to his own skin or to a third party's skin. Element 116 represents an apparatus for transferring said skin care product to said user, which in this embodiment refers to a box used to contain said liquid soap 110a. Element 118 represents an apparatus for transferring said separate ingredient to said user, which in this embodiment refers to a box used to contain said sea salt 112. Element 122 represents a customized solution that is created when said user 120 combines a desired amount of said Skin Care Product 110 with a desired amount of said separate Ingredient 112, which in this embodiment refers to a solution consisting of said liquid cleanser 110 and said sea salt 112. FIG. 1 illustrates the liquid cleanser 110a, the sea salt 112a, and the instructions for combining the two 114 being provided to the user 120. The user then combines a desired amount of the liquid cleanser 110a with a desired amount of sea salt 112a, and in doing so creates a customized solution 122. FIG. 1 also shows that the unused portion of said liquid cleanser 110b and unused sea salt 112b remain unmixed so the user may repeat the process in the future and combine a desired amount of skin cleanser 110b and sea salt 112b to create another custom solution 122 with a customized product to ingredient ratio.

### Conclusion, Ramifications, and Scope

While my above description contains many specificities, these should not be construed as limitations on the scope, but rather as an exemplification of one preferred embodiment thereof. Many other variations are possible.

The definitions mentioned in the claims of the various parts of the present device make a number of variations and combinations possible. It is important to note the skin care product need not serve the same function as the separate ingredient. Also, it is not necessary for the product and ingredient to share the same form.

For instance, in one embodiment, the skin care product may come in the form of a cream and may serve to protect the skin from ultraviolet radiation from the sun, while the separate ingredient may come in the form of oil and may serve to moisturize the skin. The means for combining the cream and oil may come in the form of instructions that encourage the user to combine a desired amount of oil with a desired amount of cream in the user's hand before applying the mixture to the skin. The instructions may be written on the side of a box that the bottle of cream and bottle of oil are packaged together in.

In another embodiment the topical skin care product may consist of a liquid skin cleanser that contains some alcohol-based astringent, while the separate ingredient consists of some separate alcohol-based astringent. The means for combining the cleanser and separate astringent may consist of instructions printed on the container that encourage the user to mix the toner and astringent together.

Another embodiment may consist of a topical skin care product, such as a skin cleanser, moisturizing lotion, astringent, or some other topical skin care product that does not contain any amount of the separately included ingredient, along with a separate container of one or a combination of exfoliating ingredient, moisturizing ingredient, or some other ingredient or ingredients any combination of the aforementioned.

Another embodiment may consist of a container of topical skin care product that contains some amount of the included separate ingredient already mixed in with the skin care product, along with a separate container of that same ingredient that may be combined with the topical skin care product by the user to modify the product-ingredient ratio.

Another embodiment may consist of a topical skin care product along with a separate ingredient that may be combined with the topical skin care product along with a container that may be used to combine the products before every use.

In one embodiment, the topical skin care product solution may contain some of the separate ingredient, or it may contain none of the additional ingredient.

In one embodiment, more than one separate ingredient may be provided along with the topical skin care product to enable the user to modify more than one property of the skin care product.

A user controlled customizable skin care product with variable product-to-ingredient ratio comprising:

1. A topical skin care product.

2. The topical skin care product of claim 1 wherein said topical skin care product refers to an element, compound, or mixture that may be applied to the skin or hair that consists of a lotion, shake lotion, cream, ointment, gel, foam, dermal patch, dermal mask, powder, solid, liquid, gas, vapor, sponge, tape, paste, tincture, or combination of the aforementioned.

3. The topical skin care product of claim 1 wherein said topical skin care product refers to any element, compound, or mixture that may be applied to the skin or hair in order to cleanse, clean, moisturize, dry, tone, exfoliate, medicate, treat medical condition of, strengthen, grow, reverse aging of, alter elasticity of, nourish, numb, shave, remove, cool, heat, alter sensation of, soften, harden, protect from the elements, protect from radiation, protect from foreign objects, lubricate, alter volume of, prevent from sunburn, irritate, or decrease irritation of the skin or hair.

4. The topical skin care product of claim 1 wherein said topical skin care product is contained in an apparatus for transferring said skin care product to said user.

5. The apparatus of claim 4 wherein said apparatus refers to any device or container that can be used to house, store, contain, wrap, cover, or transport said skin care product to facilitate transfer of said skin care product to the user.

6. At least one separate ingredient that may be combined with said topical skin care product by said user.

7. The separate ingredient of claim 6 wherein said separate ingredient refers to an element, compound, or mixture that may be applied to the skin or hair that consists of a lotion, shake lotion, cream, ointment, gel, foam, dermal patch, dermal mask, powder, solid, liquid, gas, vapor, sponge, tape, paste, tincture, or combination of the aforementioned.

8. The separate ingredient of claim 6 wherein said separate ingredient refers to any element, compound, or mixture that may be applied to the skin or hair in order to cleanse, clean, moisturize, dry, tone, exfoliate, medicate, treat medical condition of, strengthen, grow, reverse aging of, alter elasticity of, nourish, numb, shave, remove, cool, heat, alter sensation of, soften, harden, protect from the elements, protect from radiation, protect from foreign objects, lubricate, alter volume of, prevent from sunburn, irritate, or decrease irritation of the skin or hair.

9. The separate ingredient of claim 6 wherein said separate ingredient is contained in an apparatus for transferring said skin care product to said user.

10. The apparatus of claim 9 wherein said apparatus refers to any device or container that can be used to house, store, contain, wrap, cover, or transport said separate ingredient to facilitate transfer of said separate ingredient to the user.

11. A means for the user to combine said separate ingredient with said topical skin care product.

12. The means of claim 11 wherein said means refers to any instructions, written text, message, symbols, symbolic placement of said skin care product or said separate ingredient or combination of the aforementioned that encourages the user to combine said topical skin care product with said separate ingredient in a way that allows the user to customize the product-ingredient ratio.

13. The user of claim 11 wherein said user refers to any individual who Intends to apply said skin care product to his own skin or hair or to the skin or hair of a third party.
